(19) United States
(12) Patent Application Publication
(10) Pub. No.: US 2016/0319264 A1
(43) Pub. Date: Nov. 3, 2016

(54) Yield Improvement by PH-Stabilization of Enzymes

(71) Applicant: NOVOZYMES A/S, Bagsvaerd (DK)

(72) Inventor: Tomoko Matsui, Chiba-Shi (JP)

(73) Assignee: Novozymes A/S, Bagsvaerd (DK)

(21) Appl. No.: 15/108,344

(22) PCT Filed: Jan. 8, 2015
(86) PCT No.: PCT/EP2015/050236

§ 371 Date: Jun. 27, 2016

(30) Foreign Application Priority Data
Jan. 13, 2014 (EP) 14150949.7

(51) Int. Cl. C12N 9/44; C12Q 1/40; C12N 9/96

(52) U.S. Cl. CPC C12N 9/2457; C12N 9/96; C12Y 302/01041; C12Q 1/40; G01N 2333/944

(57) ABSTRACT

The invention relates to methods of improving the yield or productivity of a variant enzyme derived from a parent enzyme, said method comprising the step of selecting a host cell that produces a variant enzyme which has at least the specific enzymatic activity of the parent enzyme as well as an improved pH-stability in the pH range of 5-9.

### REFERENCE TO SEQUENCE LISTING

This application contains a Sequence Listing in computer readable form. The computer readable form is incorporated herein by reference.

### FIELD OF THE INVENTION

The invention relates to methods of improving the yield or productivity of an enzyme variant derived from a parent enzyme, said method comprising the step of selecting a host cell that produces an enzyme variant which has at least the specific enzymatic activity of the parent enzyme as well as an improved pH-stability in the pH range of 5-9.

### BACKGROUND OF THE INVENTION

Enzymes have been protein engineered in order to generate artificial variants for quite some time to provide or adjust certain properties of interest, such as, pH dependent activity, thermostability, substrate cleavage pattern, specific activity of cleavage, substrate specificity and/or substrate binding (WO0151620A2).

The identification of a screening property that would be suitable as a target for protein engineering to improve the yield or productivity of an enzyme of interest would be highly interesting for the industrial manufacture of enzymes.

### SUMMARY OF THE INVENTION

In the examples provided herein it was surprisingly shown, that an increase in the pH stability of an enzyme, i.e., an increase in the ability of an enzyme to retain its activity after some duration at a certain pH level, correlated with a significant improvement in yield and/or productivity.

Accordingly, in a first aspect the invention provides methods of improving the yield and/or productivity of an enzyme, said method comprising the steps of:
a) providing a host cell comprising an expression gene library of mutated polynucleotides encoding one or more variant of a parent enzyme of interest, wherein the one or more variant comprises at least one amino acid alteration compared to the parent enzyme;
b) cultivating the host cell under conditions conducive for the production of the one or more variant enzyme;
c) selecting a host cell that produces a variant enzyme which has at least the specific enzymatic activity of the parent enzyme as well as an improved pH-stability in the pH range of 5-9, wherein the yield and/or productivity of the variant enzyme is improved compared to that of the parent; and optionally
d) recovering the variant enzyme.

### BRIEF DESCRIPTION OF DRAWINGS

FIG. 1 shows the pullulanase expression level of Bacillus subtilis MDT99, a very low-protease (delta-11) strain comprising the chromosomally integrated parent pullulanase-encoding gene of Example 1, compared with that of Bacillus subtilis HyGe380, the same background host strain with the chromosomally integrated Variant8-encoding gene. The results show that Variant8 has an improved expression.

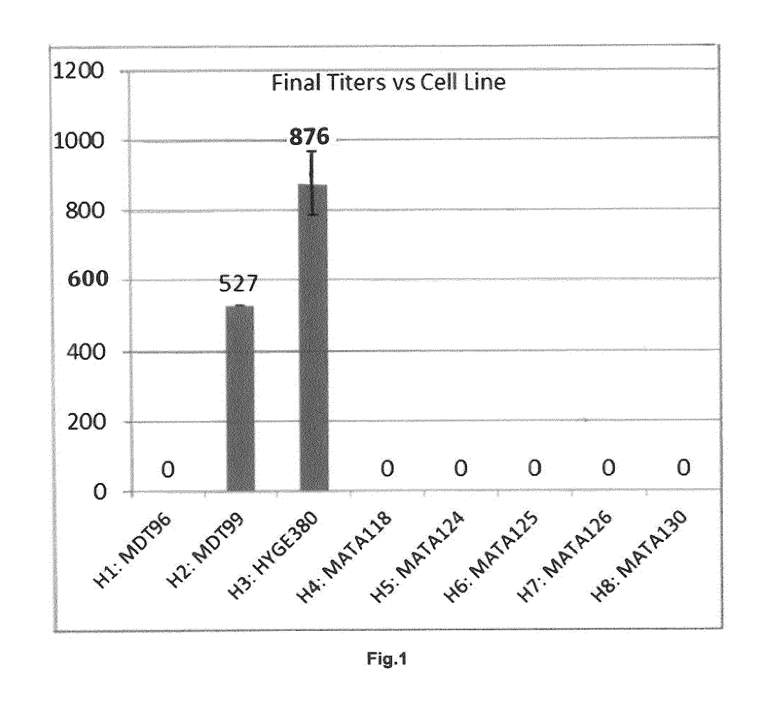

### DETAILED DESCRIPTION OF THE INVENTION

The first aspect of the invention provides methods of improving the yield and/or productivity of an enzyme, said method comprising the steps of:
a) providing a host cell comprising an expression gene library of mutated polynucleotides encoding one or more variant of a parent enzyme of interest, wherein the one or more variant comprises at least one amino acid alteration compared to the parent enzyme;
b) cultivating the host cell under conditions conducive for the production of the one or more variant enzyme;
c) selecting a host cell that produces a variant enzyme which has at least the specific enzymatic activity of the parent enzyme as well as an improved pH-stability in the pH range of 5-9, wherein the yield and/or productivity of the variant enzyme is improved compared to that of the parent; and optionally
d) recovering the variant enzyme.

Coding sequence: The term “coding sequence” or “polynucleotide encoding” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Mutated polynucleotides: Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 1, SEQ ID NO: 3 or SEQ ID NO: 15, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for enzyme activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

### Sources of Polypeptides Having Enzyme Activity

A polypeptide having enzyme activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a bacterial polypeptide. For example, the polypeptide may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces polypeptide having [enzyme] activity, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma polypeptide.

In one aspect, the polypeptide is a Bacillus acidopullulyticus, Bacillus alkalophilus, Bacillus amyloliquefaciens, Bacillus brevis, Bacillus circulans, Bacillus clausii, Bacillus coagulans, Bacillus deramificans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide.

In another aspect, the polypeptide is a Streptococcus equisimilis, Streptococcus pyogenes, Streptococcus uberis, or Streptococcus equi subsp. Zooepidemicus polypeptide.

In another aspect, the polypeptide is a Streptomyces achromogenes, Streptomyces avermitilis, Streptomyces coelicolor, Streptomyces griseus, or Streptomyces lividans polypeptide.

The polypeptide may be a fungal polypeptide. For example, the polypeptide may be a yeast polypeptide such as a Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide.

In another aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

In a preferred embodiment, the parent enzyme is an enzyme selected from the group consisting of hydrolase, isomerase, ligase, lyase, oxidoreductase, or transferase; preferably the parent enzyme is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, pullulanase, ribonuclease, transglutaminase, or xylanase; more preferably the parent enzyme is a pullulanase GH13_14; and most preferably the parent enzyme is a pullulanase from a Bacillus sp.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Preferably, the parent enzyme is a pullulanase encoded by a polynucleotide having at least 60% sequence identity to the polynucleotide sequence of SEQ ID NO:1, SEQ ID NO:3 or SEQ ID NO:15 and/or the parent enzyme is a pullulanase having at least 60% sequence identity to the polypeptide sequence of SEQ ID NO:2, SEQ ID NO:4 or SEQ ID NO:16. Preferably, the parent enzyme is a pullulanase encoded by a polynucleotide having at least 65% sequence identity to the polynucleotide sequence of SEQ ID NO:1, SEQ ID NO:3 or SEQ ID NO:15; or more preferably at least 70% sequence identity, 75%, 80%, 85%, 90%, 95%, 97%, 98% or 99% sequence identity. Preferably, the parent enzyme is a pullulanase having at least 60% sequence identity to the polypeptide sequence of SEQ ID NO:2, SEQ ID NO:4 or SEQ ID NO:16; or more preferably at least 70% sequence identity, 75%, 80%, 85%, 90%, 95%, 97%, 98% or 99% sequence identity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus acidopullulyticus, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

In a preferred embodiment, the host cell is a prokaryotic host cell, preferably a Gram-positive bacterium; more preferably a Gram-positive bacterium of a Genus selected from the group consisting of Bacillus, Clostridium, Enterococcus, Geobacillus, Lactobacillus, Lactococcus, Oceanobacillus, Staphylococcus, Streptococcus, and Streptomyces; and most preferably the host cell is of a species selected from the group consisting of Bacillus acidopullulyticus, Bacillus alkalophilus, Bacillus amyloliquefaciens, Bacillus brevis, Bacillus circulans, Bacillus clausii, Bacillus coagulans, Bacillus deramificans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis.

### Expression Vectors

The present invention also relates to recombinant expression vectors, constructs or gene libraries comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis crylllA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis crylllA gene (WO 94/25612) and a Bacillus subtilis SP82 gene (Hue et al., 1995, Journal of Bacteriology 177: 3465-3471).

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. Other examples of regulatory sequences are those that allow for gene amplification.

The method of the invention may be employed in an iterative manner, where the variant enzyme of step (d) in one cycle is used as the starting point or parent enzyme in the subsequent cycle. Preferably, steps (a) to (d) are repeated at least once, wherein the variant enzyme in step (d) of each cycle or repetition serves as the parent enzyme in the subsequent cycle.

### Improved Yield

The terms “improved yield” or “improved productivity” in the context of the present invention means that the final amount of product produced per added amount of substrate is improved or that the same amount of product is obtained by a shorter cultivating period.

Preferably, the yield and/or productivity of the variant enzyme is improved by at least 10%; preferably by at least 20%; more preferably by at least 30%; still more preferably by at least 40% and most preferably by at least 50%—preferably in each cycle.

It is also preferred that the pH-stability of the variant enzyme in the pH range of 5-9 is improved by at least 10%; preferably by at least 20%, 30%, 40%, 50%, 60%, 70%, 80%, 90%, or 100%; and most preferably by at least 120%—preferably in each cycle.

Further, it is preferred that the pH-stability of the variant enzyme is determined as in Example 3 herein and is improved by at least 10%; preferably by at least 20%, 30%, 40%, 50%, 60%, 70%, 80%, 90%, or 100%; and most preferably by at least 120%—preferably in each cycle.

In addition, it is preferred that the variant enzyme has an improved pH stability at pH 7 and/or 8 determined as in Example 4 herein over the parent enzyme; preferably improved by at least 10%; preferably by at least 20%, 30%, 40%, 50%, 60%, 70%, 80%, 90%, or 100%; and most preferably by at least 120%.

Preferably the variant enzyme has an improved pH stability in the pH range of 6-8; more preferably in the pH range of 6-7 or in the pH range of 6.5-7.5.

In a preferred embodiment, the variant enzyme has an improved pH stability at pH 5, at pH 6, at pH 7, at pH 8 or at pH 9; most preferably the variant enzyme has an improved pH stability at pH 7 determined as in Example 3 or 4 herein over its parent enzyme.

Finally, it is preferred that the variant enzyme has an improved pH-stability in the pH range of 5-9; preferably in the pH range of 6-9; more preferably the pH range of 6.5-9; more preferably the pH range of 7-9; and most preferably the pH range of 7-8.

### EXAMPLES

### Example 1

### Pullulanase Assay

### Red-Pullulan Assay (Megazyme)

### Substrate Solution

0.1 g red-pullulan (Megazyme)

15 ml 50 mM sodium acetate, pH5

A reaction mixture was prepared by mixing 10 μl of an enzyme sample together with 80 μl of substrate soln. and incubated at 55° C. for 20 min. 50 μl of ethanol was added to the reaction mixture and centrifuged for 10 min. at 3500 rpm. The supernatants were carefully taken out and the absorbance at A510 was read.

A commercial pullulanase enzyme, Promozyme® D2 (Novozymes), was used as standard to determine pullulanase activity units in the Megazyme pullulanase assay according to the manufacturers instructions.

### Example 2

### Construction of Chimera Pullulanase Variants

Genomic DNAs encoding pullulanase from Bacillus acidopullulyticus NCIB11777 (SEQ ID NO:1 encoding SEQ ID NO:2) and Bacillus deramificans (SEQ ID NO:3 encoding SEQ ID NO:4) under the control of a triple promoter system (as described in WO 99/43835) consisting of the promoters from Bacillus licheniformis alpha-amylase gene (amyI), Bacillus amyloliquefaciens alpha-amylase gene (amyQ), and the Bacillus thuringiensis crylllA promoter including stabilizing sequence were isolated using NucleoSpin® Tissue kit [MACHEREY-NAGEL according to the manufacturers instructions. The gene coding for Chloramphenicol acetyltransferase (CAT) is associated with the pullulanase gene cassette (Described in e.g. Diderichsen, B; Poulsen, G. B.; Joergensen, S. T.; A useful cloning vector for Bacillus subtilis. Plasmid 30:312(1993)) and used as a selective marker (Diderichsen et al., 1983, Plasmid 30:312-315).

The genomes contain pullulanase genes as shown in SEQ ID NO:1 and SEQ ID NO:3, respectively. The genomic DNAs were used as templates for PCR amplification, which was carried out using the below FORWARD primer and reverse primers (variant 1 R-8R) under the following conditions.

PCR1 conditions

| | |
|---|---|
| 1.0 μl | Template |
| 4.8 μl | H₂O |
| 4 μl | Phusion HF Buffer |
| 1.6 μl | dNTP (2.5 mM) |
| 0.2 μl | FORWARD and reverse primers (20 μM) |
| 0.4 μl | Phusion ® High-Fidelity DNA Polymerase (ThermoScientific) |

98° C./30 sec

30× (98° C./10 sec, 60° C./20 sec, 72° C./3 min)

72° C./5 min

FORWARD PRIMER
(SEQ ID NO: 5)
cggaacgcctggctgacaacacg
Variant1R
(SEQ ID NO: 6)
atccaaatacgcattcgaaacagcagccgatgcgatcgatgaac
Variant2R
(SEQ ID NO: 7)
ctgtaataataacgaggcaaattaagcacattacgagatatcac
Variant3R
(SEQ ID NO: 8)
catccaggaggattcgtatcttccaggtccacaatcatgcctctc
Variant4R
(SEQ ID NO: 9)
gttaccatcgagtccgttccgaatattgtcattaaacaccgctac
Variant5R
(SEQ ID NO: 10)
tctaaataagcgttgcttacagcctttggagtcgctgcagcctg
Varian6R
(SEQ ID NO: 11)
ctgtgatgaatcaagcacattacgtggtatgagattgactgcttc
Variant7R
(SEQ ID NO: 12)
caggtccacaatcatgcctctcgttgcattgactgaaatagcacg
Variant8R
(SEQ ID NO: 13)
gtttcgtaaattgtcattaaacacgccaattcccaagcccttttg
REVERSE PRIMER
(SEQ ID NO: 14)
caatccaagagaaccctgatacggatg

PCR fragments were isolated in 0.7% agarose gel and recovered by Qiagen Gel extraction kit and then the 2^{nd} PCR amplification was carried out using the first PCR fragment as a forward primer and REVERSE PRIMER using Bacillus NCIB11777's genome for variant 1-4 and B. deramificans NN18718's genome for variant 5-8 as templates.

PCR2 conditions

| | |
|---|---|
| 0.6 μl | template |
| 0.3 μl | REVERSE primer(20 μM) |
| 3 μl | Phusion HF Buffer |
| 1.56 μl | dNTP (2.5 mM) |
| 0.36 μl | Phusion ® High-Fidelity DNA Polymerase (ThermoScientific) |
| 5.18 μl | H2O |
| 4.0 μl | Mega-primer (150 ng fragment from PCR1) |

98° C./5 min

10× (98° C./30 sec, 68° C./15 sec, 72° C./6 min)

25× (98° C./30 sec, 60° C./5 sec, 72° C./6 min)

72° C./10 min

The resultant PCR fragments having pullulanase gene with Bacillus genome flanking regions and CAT gene were integrated into B. subtilis host cell genome.

### Example 3

### Screening for Improved pH Stability by MTP Cultivation

Bacillus libraries or variants were cultivated in MTP containing two cultivation media, medium 1 and medium 2, whose final pHs after 2 or 3 days cultivation are around 8 and 6-7, respectively.

Medium 1; pH approx. 8

| | |
|---|---|
| Bacto ™ Tryptone | 20 g/L |
| Bacto ™Yeastextract | 5 g/L |
| FeCl₂ 6H₂O | 0.007 g/L |
| MnCl₂ 4H₂O | 0.003 g/L |
| MgSO₄ 7H₂O | 0.015 g/L |

Medium 2; pH approx. 6-7

| | |
|---|---|
| Bacto ™ Tryptone | 13.3 g/L |
| Bacto ™ Yeast extract | 26.6 g/L |
| Glycerol | 4.4 g/L |

Pullulanase activity was measured by red-pullulan assay described in Example 1 and the ratios of the productivity between medium 1 and medium 2 were determined; the ratios are not influenced by changes in the specific activity. Variants having higher medium 1/medium 2 ratio than the parent pullulanase were selected as pH stability-improved candidates. See table 1 for results. Variant8 was selected for further study, the encoding DNA sequence is provided in SEQ ID NO: 15 and the encoded amino acid sequence in SEQ ID NO: 16.

TABLE 1
Improved pH stability expressed as ratio of pullulanase
productivity of variants in medium1 versus in medium2.

| | Par- | Vari- | Vari- | Vari- | Vari- | Vari- | Vari- | Vari- | Vari- |
|---|---|---|---|---|---|---|---|---|---|
| Ratio | ent | ant 1 | ant 2 | ant3 | ant4 | ant 5 | ant 6 | ant7 | ant8 |
| Medi- | 55% | 27% | 14% | 26% | 20% | 117% | 109% | 93% | 130% |
| um1/ | | | | | | | | | |
| medi- | | | | | | | | | |
| um2 | | | | | | | | | |

### Example 4

### Screening for pH Stability

The residual activities after incubating in assay buffers; 50 mM succinic acid, 50 mM HEPES, 50 mM CHES, 50 mM CABS, 1 mM CaCl2, 75 mM KCl, 0.01% Triton X-100, complete protease inhibitor cocktail (Roche Applied Science) adjusted to pH-values 6.0, 7.0 and 8.0 with HCl or NaOH, at 55° C. for 30 minutes were measured using red-pullulan assay described in Example 1. The variants were confirmed to have improved pH stability over the parent at pH 7 and/or 8, as shown in table 2.

TABLE 2
pH stability after 30 minutes at 55° C. at pH 6, 7 and 8.

| | pH6 | pH7 | pH8 |
|---|---|---|---|
| Parent | 100% | 16% | 10% |
| Variant6 | 42% | 42% | 10% |
| Variant8 | 93% | 93% | 45% |

### Example 5

### Construction of B. subtilis Expression Hosts

B. subtilis MDT191 is a very low-protease host strain. It was derived from B. subtilis A164 (ATCC 6051A) by introduction of deletions in the following genes: sigF (spollAC), nprE, aprE, amyE, srfAC, wprA, bpr, vpr, mpr, epr, and ispA.

MDT191 was transformed with about 1 μg Variant8 genomic DNA according to the procedure of Anagnostopoulos and Spizizen (J. Bacteriol. 1961. 81:741-746)

Chloramphenicol resistant transformants were checked for pullulanase activity as follows. Transformants were patched on Difco Tryptose Blood Agar Base (BD Diagnostics, Franklin Lakes, N.J., USA)+5 μg/ml chloramphenicol, along with JPUL-008 and MDT191 patched on LB plate. The plates were incubated at 37° C. overnight. Then the plates were overlaid by 1% agar+0.5% Remazol brilliant blue-dyed pullulan and 100 mM Na acetate. The plates were incubated at 50° C. for several hours. Both Variant8 positive controls and the transformants made clearing zones in the Remazol brilliant blue-dyed pullulan, indicating pullulanase activity, while the host MDT191 negative control did not. One transformant was selected named B. subtilis HyGe380.

### Example 6

### Expression Evaluation in Jar Fermentation

The B. subtilis strains expressing the parent pullulanases as well as Variant 8 (HyGe380) were fermented in 1 L jars. Each Bacillus strain was cultivated in the medium containing glucose, ammonium sulfate, dipotassium phosphate, disodium phosphate, magnesium sulfate and metals at 37° C., pH6.5 in 1 L lab fermenters with adequate agitation and aeration for 3 days.

Culture aliquots were taken periodically during fermentation. The samples were centrifuged and the supernatants were used to measure pullulanase productivities by red-pullulan assay described in EXAMPLE 1.

Their productivities are listed in the below table. The supernatants were also run in SDS-PAGE (ATTO e-PAGEL 12.5%) and they were confirmed to have the strength of the band signal corresponding to measured activity units.

TABLE 3
Pullulanase productivities of the parent and chimeric Variant8 pullulanase.

| | 30 h | 50 h |
|---|---|---|
| Parent | 18 U/ml | 50 U/ml |
| Variant 8 | 45 U/ml | 100 U/ml |

### Example 7

### Construction of Pullulanase Libraries

PCR was carried out using FORWARD primer shown below having at least 15 mer homologous to the vector flanking and N-terminal pullulanase sequence, and a reverse mutation primer having saturation mutagenesis at one or two sites with the genomic DNA of variant 8 as a template. Another PCR was carried out using a forward primer having at least 15 mer homologous to the region of a paired reverse mutation primer and REVERSE primer shown below having at least 15 mer homologous to the vector flanking. Designing of primers was followed to In-Fusion cloning procedure (CLONETECH).
FORWARD primer ttgcttttagttcatcgatagcatcagcagattctacctcgacagaag (SEQ ID NO:17)
REVERSE primer ttattgattaacgcgtttactttttaccgtggtctg (SEQ ID NO:18)

PCR conditions

| | |
|---|---|
| 1.0 μl | Template |
| 4.8 μl | H₂O |
| 4 μl | Phusion HF Buffer |
| 1.6 μl | dNTP (2.5 mM) |
| 0.2 μl | FORWARD and reverse primers (20 μM) |
| 0.4 μl | Phusion ® High-Fidelity DNA Polymerase (ThermoScientific) |

98° C./30 sec

30× (98° C./10 sec, 60° C./20 sec, 72° C./3 min)

72° C./5 min

Two PCR fragments were gel-purified and cloned in an expression vector comprising the genetic elements as described in WO99/43835 by In-Fusion cloning (CLONTECH) following its user manual. By doing so, the signal peptide from the alkaline protease from Bacillus clausii (aprH) was fused to library genes as described in WO99/43835 in frame to the DNA encoding pullulanase.

The resultant In-Fusion ligation solution was transformed into E. coli DH5alpha and the library plasmids were recovered from E. coli library transformants. The plasmid library was then integrated by homologous recombination into a B. subtilis host cell genome. The gene was expressed under the control of a triple promoter system (as described in WO 99/43835). The gene coding for chloramphenicol acetyltransferase was used as maker as described in Diderichsen et al., 1993, Plasmid 30:312-315.

Library clones were cultivated in 96 well MTPs containing medium2 supplemented with 6 mg/L chloramphenicol for 1-3 days at 30-37° C. The plate was centrifuged and the culture supernatants were used for pullulanase assay.

### Example 8

### Screening for Improved pH Stability

Culture supernatants were measured for stability as described in example 4 and a clone with higher residual activity at pH7 than the parent pullulanase was selected.

TABLE 4
Pullulanase productivities of the parent
and synthetic Variant75 pullulanase.

| | Mutation | pH7 |
|---|---|---|
| Parent | | 58% |
| Variant75 | E699R | 75% |

The genomic DNAs of selected variant was isolated using NucleoSpin® Tissue kit [MACHEREY-NAGEL according to its procedure and the pullulanase coding sequence was PCR-amplified using FORWARD and REVERSE primers described in EXAMPLE 7 and then sequenced to determine its sequences.

### Example 9

### Expression Evaluation in MTPs

A screened B. subtilis clone, variant 75, described in EXAMPLE 8 was fermented in at least 4 wells containing medium 1 or medium 2 with 6 mg/L chloramphenicol at 220 rpm, 37° C. for 3 days. The cultures were centrifuged and the supernatants were carefully taken out for pullulanase assay and the average expression levels were compared to the parent pullulanase to confirm the variant having higher stability at pH7 showed higher expression levels in either medium used.

TABLE 5
Expression levels of variant75 versus its parent in different media.

| | | Medium1 | Medium 2 |
|---|---|---|---|
| Parent | | 100% | 100% |
| variant75 | E699R | 315% | 365% |

1. A method of improving the yield and/or productivity of an enzyme, said method comprising the steps of:
a) providing a host cell comprising an expression gene library of mutated polynucleotides encoding one or more variant of a parent enzyme of interest, wherein the one or more variant comprises at least one amino acid alteration compared to the parent enzyme;
b) cultivating the host cell under conditions conducive for the production of the one or more variant enzyme;
c) selecting a host cell that produces a variant enzyme which has at least the specific enzymatic activity of the parent enzyme as well as an improved pH-stability in the pH range of 5-9, wherein the yield and/or productivity of the variant enzyme is improved compared to that of the parent; and optionally
d) recovering the variant enzyme.

2. The method of claim 1, wherein the parent enzyme is an enzyme selected from the group consisting of hydrolase, isomerase, ligase, lyase, oxidoreductase, or transferase; preferably the parent enzyme is an alpha-galactosidase, alpha-glucosidase, am inopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, pullulanase, ribonuclease, transglutaminase, or xylanase.

3. The method of claim 1, wherein the parent enzyme is a pullulanase encoded by a polynucleotide having at least 60% sequence identity to the polynucleotide sequence of SEQ ID NO:1, SEQ ID NO:3 or SEQ ID NO:15.

4. The method of claim 1, wherein the parent enzyme is a pullulanase having at least 60% sequence identity to the polypeptide sequence of SEQ ID NO:2, SEQ ID NO:4 or SEQ ID NO:16.

5. The method of claim 1, wherein the host cell is a prokaryotic host cell selected from the group consisting of Bacillus, Clostridium, Enterococcus, Geobacillus, Lactobacillus, Lactococcus, Oceanobacillus, Staphylococcus, Streptococcus, and Streptomyces; and most preferably the host cell is of a species selected from the group consisting of Bacillus acidopullulyticus, Bacillus alkalophilus, Bacillus amyloliquefaciens, Bacillus brevis, Bacillus circulans, Bacillus clausii, Bacillus coagulans, Bacillus deramificans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis.

6. The method of claim 1, wherein steps (a) to (d) are repeated at least once, and wherein the variant enzyme in step (d) of each cycle serves as the parent enzyme in the subsequent cycle.

7. The method of claim 1, wherein the yield or productivity of the variant enzyme is improved by at least 10%.

8. The method of claim 1, wherein the pH-stability of the variant enzyme in the pH range of 5-9 is improved by at least 10%.

9. The method of claim 1, wherein the pH-stability of the variant enzyme determined as in Example 3 herein is improved by at least 10%.

10. The method of claim 1, wherein the variant enzyme has an improved pH stability at pH 7 and/or 8 determined as in Example 4 herein over the parent enzyme.

11. The method of claim 1, wherein the variant enzyme has an improved pH-stability in the pH range of 5-9.


SEQUENCE LISTING (18 sequences)

SEQ ID NO: 1 - DNA, 2586 bp, Bacillus acidopullulyticus
  CDS (1)..(2583)
atgtccctaatacgttctaggtataatcattttgtcattctttttactgtcgccataatgtttctaacagtttgtttccccgcttataaagctttagcagattctacctcgacagaagtcattgtgcattatcatcgttttgattctaactatgcaaattgggatctatggatgtggccatatcaaccagttaatggtaatggagcagcatacgagttttctggaaaggatgattttggcgttaaagcagatgttcaagtgcctggggatgatacacaggtaggtctgattgtccgtacaaatgattggagccaaaaaaatacatcagacgatctccatattgatctgacaaaggggcatgaaatatggattgttcagggggatcccaatatttattacaatctgagtgatgcgcaggctgcagcgactccaaaggtttcgaatgcgtatttggataatgaaaaaacagtattggcaaagctaactaatccaatgacattatcagatggatcaagcggctttacggttacagataaaacaacaggggaacaaattccagttaccgctgcaacaaatgcgaactcagcctcctcgtctgagcagacagacttggttcaattgacgttagccagtgcaccggatgtttcccatacaatacaagtaggagcagccggttatgaagcagtcaatctcataccacgaaatgtattaaatttgcctcgttattattacagcggaaatgatttaggtaacgtttattcaaataaggcaacggccttccgtgtatgggctccaactgcttcggatgtccaattacttttatacaatagtgaaacaggacctgtaaccaaacagcttgaaatgcaaaagagtgataacggtacatggaaactgaaggtccctggtaatctgaaaaattggtattatctctatcaggtaacggtgaatgggaagacacaaacagccgttgacccttatgtgcgtgctatttcagtcaatgcaacacgtggtatgatagtcgatttagaagatacgaatcctcctggatggaaagaagatcatcaacagacacctgcgaacccagtggatgaagtaatctacgaagtgcatgtgcgtgatttttcgattgatgctaattcaggcatgaaaaataaagggaaatatcttgcctttacagaacatggcacaaaaggccctgataacgtgaaaacgggtattgatagtttgaaggaattaggaatcaatgctgttcaattacagccgattgaagaatttaacagcattgatgaaacccaaccaaatatgtataactggggctatgacccaagaaactacaacgtccctgaaggagcgtatgcaactacaccagaaggaacggctcgcattacccagttaaagcaactgattcaaagcattcataaagatcggattgctatcaatatggatgtggtctataaccatacctttaacgtaggagtgtctgattttgataagattgttccgcaatactattatcggacagacagcgcaggtaattatacgaacggctcaggtgtaggtaatgaaattgcgaccgagcgtccgatggtccaaaagttcgttctggattctgttaaatattgggtaaaggaataccatatcgacggcttccgtttcgatcttatggctcttttaggaaaagacaccatggccaaaatatcaaaagagcttcatgctattaatcctggcattgtcctgtatggagaaccatggactggcggtacctctggattatcaagcgaccaactcgttacgaaaggtcagcaaaagggcttgggaattggcgtattcaacgataatattcggaacggactcgatggtaacgtttttgataaatcggcacaaggatttgcaacaggagatccaaaccaagttaatgtcattaaaaatggagttatgggaagtatttcagatttcacttcggcacctagcgaaaccattaactatgtaacaagccatgataatatgacattgtgggataaaattagcgcaagtaatccgaacgatacacaagcagatcgaattaagatggatgaattggctcaagctgtggtatttacttcacaaggggtaccatttatgcaaggtggagaagaaatgctgcggacaaaaggcggtaatgataatagttacaatgccggggatagcgtgaatcagttcgattggtcaagaaaagcacaatttgaaaatgtattcgactactattcttggttgattcatctacgtgataatcacccagcattccgtatgacgacagcggatcaaatcaaacaaaatctcactttcttggatagcccaacgaacactgtagcatttgaattaaaaaatcatgccaatcatgataaatggaaaaacattatagttatgtataatccaaataaaactgcacaaactctcactctaccaagtggaaattggacaattgtaggattaggcaatcaagtaggtgagaaatcactaggccatgtaaatggcacggttgaggtgccagctcttagtacgatcattcttcatcagggtacatctgaagatgtcattgatcaaaat

SEQ ID NO: 2 - protein, 861 aa, Bacillus acidopullulyticus
MSLIRSRYNHFVILFTVAIMFLTVCFPAYKALADSTSTEVIVHYHRFDSNYANWDLWMWPYQPVNGNGAAYEFSGKDDFGVKADVQVPGDDTQVGLIVRTNDWSQKNTSDDLHIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIQVGAAGYEAVNLIPRNVLNLPRYYYSGNDLGNVYSNKATAFRVWAPTASDVQLLLYNSETGPVTKQLEMQKSDNGTWKLKVPGNLKNWYYLYQVTVNGKTQTAVDPYVRAISVNATRGMIVDLEDTNPPGWKEDHQQTPANPVDEVIYEVHVRDFSIDANSGMKNKGKYLAFTEHGTKGPDNVKTGIDSLKELGINAVQLQPIEEFNSIDETQPNMYNWGYDPRNYNVPEGAYATTPEGTARITQLKQLIQSIHKDRIAINMDVVYNHTFNVGVSDFDKIVPQYYYRTDSAGNYTNGSGVGNEIATERPMVQKFVLDSVKYWVKEYHIDGFRFDLMALLGKDTMAKISKELHAINPGIVLYGEPWTGGTSGLSSDQLVTKGQQKGLGIGVFNDNIRNGLDGNVFDKSAQGFATGDPNQVNVIKNGVMGSISDFTSAPSETINYVTSHDNMTLWDKISASNPNDTQADRIKMDELAQAVVFTSQGVPFMQGGEEMLRTKGGNDNSYNAGDSVNQFDWSRKAQFENVFDYYSWLIHLRDNHPAFRMTTADQIKQNLTFLDSPTNTVAFELKNHANHDKWKNIIVMYNPNKTAQTLTLPSGNWTIVGLGNQVGEKSLGHVNGTVEVPALSTIILHQGTSEDVIDQ

SEQ ID NO: 3 - DNA, 2466 bp, Bacillus deramificans
  CDS (1)..(2463)
  misc_feature (1)..(3): Since this is a 5′ truncated coding sequence, the first codon does NOT encode a methionine but an alanine.
gctgtaagcaacgcttatttagatgcttcaaaccaagttttagttaagcttagccaaccgtttacactcggtgaaggagcaagcggcttcacggttcatgatgacaccgtaaataaggatatcccagtgacatctgtgacggatgcaagtcttggtcaaaatgtaaccgctgttttggcaggtaccttccaacatatttttggaggttccgattgggcacctgataatcacagtactttattaaaaaaggtgaataacaatctctatcaattctcaggagatcttcctgaaggaaactaccaatataaagtggctttaaatgatagctggaataatccgagttacccttcaaacaatatcgatttaaccgtaccaacaggcggtgcccatgtcaccttttcctatgtcccctcaacgcatgccgtctacgacagtattaacaaccctggcgccgatttacctgtaaatggcagcggggttaaaacggatctcgtgacggttactctaggggaagatccagatgtgagccatactctgtccattcaaacagatggctatcaagcaaagcaggtgatatctcgtaatgtgcttgattcatcacagtattactattcaggagatgatcttggaaatacctatacacataaagcaactacctttaaggtctgggcacctacttctactcaagtaaatgttcttctttataatagtgcaacgggttctgtaacaaaaaccgtacctatgacggcatcgggccatggtgtgtgggaagcaacggttaatcaaaaccttgaaaattggtattacatgtatgaggtaacaggccaaggctctacccgaacggctgttgatccttatgcaactgcgattgcaccaaatggaacgagaggcatgattgtggacctggctaaaacagatcctgctggctggaacagtgataaacatattacgccaaagaatatagaagatgaggtcatctatgaaatggatgtccgtgacttttccattgaccctaattcgggtatgaaaaataaagggaagtatttggctcttacagaaaaaggaacaaagggccctgacaacgtaaagacggggatagattccttaaaacaacttgggattactcatgttcagcttatgcctgttttcgcatttaacagtgtcgatgaaactgatccaacccaagataattggggttatgaccctcgcaactatgatgttcctgaagggcagtatgctacaaatgcgaatggtacggctcgtataaaagagtttaaggaaatggttctttcactccatcgtgaacacattggggttaacatggatgttgtctataatcatacctttgccacgcaaatctctgacttcgataaaattgtaccagaatattattaccgtacggatgatgcaggtaattataccaacggatcaggtactggaaatgaaatcgcagccgaaaggccaatggttcaaaaatttattattgattcccttaagtattgggtcaatgagtatcatattgacggcttccgttttgacttaatggcgctgcttggaaaagacacgatgtcgaaagctgcctcggagcttcatgctattaatccaggaattgcactttacggtgagccatggacgggtggaacctctgcactgccagaagatcagcttctgacaaaaggagctcaaaaaggcatgggagtagcggtgtttaatgacaatttacgaaacgcgttggacggcaatgtctttgattcttccgctcaaggttttgcgacaggtgcaacaggcttaactgatgcaattaagaatggcgttgaggggagtattaatgactttacctcttcaccaggtgagacaattaactatgtcacaagtcatgataactacaccctttgggacaaaatagccctaagcaaccctaatgattccgaagcggatcggattaaaatggatgaactcgcacaagcagttgttatgacctcacaaggtgttccattcatgcaaggcggggaagaaatgcttcgtacaaaaggcggcaacgacaatagttataatgcaggcgatacggtcaatgagtttgattggagcaggaaagctcaatatccagatgttttcaactattatagcgggctaatccaccttcgtcttgatcacccagccttccgcatgacgacagctaatgaaatcaatagccacctccaattcctaaatagtccagagaacacagtggcctatgaattaactgatcatgttaataaagacaaatggggaaatatcattgttgtttataacccaaataaaactgcagcaaccattaatttgccgagcgggaaatgggcaatcaatgctacgagcggtaaggtaggagaatccacccttggtcaagcagagggaagtgtccaagtaccaggtatatctatgatgatccttcatcaagaggtaagcccagaccacggtaaaaagtaa

SEQ ID NO: 4 - protein, 821 aa, Bacillus deramificans
AVSNAYLDASNQVLVKLSQPFTLGEGASGFTVHDDTVNKDIPVTSVTDASLGQNVTAVLAGTFQHIFGGSDWAPDNHSTLLKKVNNNLYQFSGDLPEGNYQYKVALNDSWNNPSYPSNNIDLTVPTGGAHVTFSYVPSTHAVYDSINNPGADLPVNGSGVKTDLVTVTLGEDPDVSHTLSIQTDGYQAKQVISRNVLDSSQYYYSGDDLGNTYTHKATTFKVWAPTSTQVNVLLYNSATGSVTKTVPMTASGHGVWEATVNQNLENWYYMYEVTGQGSTRTAVDPYATAIAPNGTRGMIVDLAKTDPAGWNSDKHITPKNIEDEVIYEMDVRDFSIDPNSGMKNKGKYLALTEKGTKGPDNVKTGIDSLKQLGITHVQLMPVFAFNSVDETDPTQDNWGYDPRNYDVPEGQYATNANGTARIKEFKEMVLSLHREHIGVNMDVVYNHTFATQISDFDKIVPEYYYRTDDAGNYTNGSGTGNEIAAERPMVQKFIIDSLKYWVNEYHIDGFRFDLMALLGKDTMSKAASELHAINPGIALYGEPWTGGTSALPEDQLLTKGAQKGMGVAVFNDNLRNALDGNVFDSSAQGFATGATGLTDAIKNGVEGSINDFTSSPGETINYVTSHDNYTLWDKIALSNPNDSEADRIKMDELAQAVVMTSQGVPFMQGGEEMLRTKGGNDNSYNAGDTVNEFDWSRKAQYPDVFNYYSGLIHLRLDHPAFRMTTANEINSHLQFLNSPENTVAYELTDHVNKDKWGNIIVVYNPNKTAATINLPSGKWAINATSGKVGESTLGQAEGSVQVPGISMMILHQEVSPDHGKK

SEQ ID NO: 5 - DNA, 23 bp, artificial sequence
  FORWARD PRIMER
cggaacgcctggctgacaacacg

SEQ ID NO: 6 - DNA, 44 bp, artificial sequence
  Variant1R
atccaaatacgcattcgaaacagcagccgatgcgatcgatgaac

SEQ ID NO: 7 - DNA, 44 bp, artificial sequence
  Variant2R
ctgtaataataacgaggcaaattaagcacattacgagatatcac

SEQ ID NO: 8 - DNA, 45 bp, artificial sequence
  Variant3R
catccaggaggattcgtatcttccaggtccacaatcatgcctctc

SEQ ID NO: 9 - DNA, 45 bp, artificial sequence
  Variant4R
gttaccatcgagtccgttccgaatattgtcattaaacaccgctac

SEQ ID NO: 10 - DNA, 44 bp, artificial sequence
  Variant5R
tctaaataagcgttgcttacagcctttggagtcgctgcagcctg

SEQ ID NO: 11 - DNA, 45 bp, artificial sequence
  Variant6R
ctgtgatgaatcaagcacattacgtggtatgagattgactgcttc

SEQ ID NO: 12 - DNA, 45 bp, artificial sequence
  Variant7R
caggtccacaatcatgcctctcgttgcattgactgaaatagcacg

SEQ ID NO: 13 - DNA, 45 bp, artificial sequence
  Variant8R
gtttcgtaaattgtcattaaacacgccaattcccaagcccttttg

SEQ ID NO: 14 - DNA, 27 bp, artificial sequence
  REVERSE PRIMER
caatccaagagaaccctgatacggatg

SEQ ID NO: 15 - DNA, 2586 bp, artificial sequence
  Chimeric variant8
atgtccctaatacgttctaggtataatcattttgtcattctttttactgtcgccataatgtttctaacagtttgtttccccgcttataaagctttagcagattctacctcgacagaagtcattgtgcattatcatcgttttgattctaactatgcaaattgggatctatggatgtggccatatcaaccagttaatggtaatggagcagcatacgagttttctggaaaggatgattttggcgttaaagcagatgttcaagtgcctggggatgatacacaggtaggtctgattgtccgtacaaatgattggagccaaaaaaatacatcagacgatctccatattgatctgacaaaggggcatgaaatatggattgttcagggggatcccaatatttattacaatctgagtgatgcgcaggctgcagcgactccaaaggtttcgaatgcgtatttggataatgaaaaaacagtattggcaaagctaactaatccaatgacattatcagatggatcaagcggctttacggttacagataaaacaacaggggaacaaattccagttaccgctgcaacaaatgcgaactcagcctcctcgtctgagcagacagacttggttcaattgacgttagccagtgcaccggatgtttcccatacaatacaagtaggagcagccggttatgaagcagtcaatctcataccacgaaatgtattaaatttgcctcgttattattacagcggaaatgatttaggtaacgtttattcaaataaggcaacggccttccgtgtatgggctccaactgcttcggatgtccaattacttttatacaatagtgaaacaggacctgtaaccaaacagcttgaaatgcaaaagagtgataacggtacatggaaactgaaggtccctggtaatctgaaaaattggtattatctctatcaggtaacggtgaatgggaagacacaaacagccgttgacccttatgtgcgtgctatttcagtcaatgcaacacgtggtatgatagtcgatttagaagatacgaatcctcctggatggaaagaagatcatcaacagacacctgcgaacccagtggatgaagtaatctacgaagtgcatgtgcgtgatttttcgattgatgctaattcaggcatgaaaaataaagggaaatatcttgcctttacagaacatggcacaaaaggccctgataacgtgaaaacgggtattgatagtttgaaggaattaggaatcaatgctgttcaattacagccgattgaagaatttaacagcattgatgaaacccaaccaaatatgtataactggggctatgacccaagaaactacaacgtccctgaaggagcgtatgcaactacaccagaaggaacggctcgcattacccagttaaagcaactgattcaaagcattcataaagatcggattgctatcaatatggatgtggtctataaccatacctttaacgtaggagtgtctgattttgataagattgttccgcaatactattatcggacagacagcgcaggtaattatacgaacggctcaggtgtaggtaatgaaattgcgaccgagcgtccgatggtccaaaagttcgttctggattctgttaaatattgggtaaaggaataccatatcgacggcttccgtttcgatcttatggctcttttaggaaaagacaccatggccaaaatatcaaaagagcttcatgctattaatcctggcattgtcctgtatggagaaccatggactggcggtacctctggattatcaagcgaccaactcgttacgaaaggtcagcaaaagggcttgggaattggcgtgtttaatgacaatttacgaaacgcgttggacggcaatgtctttgattcttccgctcaaggttttgcgacaggtgcaacaggcttaactgatgcaattaagaatggcgttgaggggagtattaatgactttacctcttcaccaggtgagacaattaactatgtcacaagtcatgataactacaccctttgggacaaaatagccctaagcaaccctaatgattccgaagcggatcggattaaaatggatgaactcgcacaagcagttgttatgacctcacaaggtgttccattcatgcaaggcggggaagaaatgcttcgtacaaaaggcggcaacgacaatagttataatgcaggcgatacggtcaatgagtttgattggagcaggaaagctcaatatccagatgttttcaactattatagcgggctaatccaccttcgtcttgatcacccagccttccgcatgacgacagctaatgaaatcaatagccacctccaattcctaaatagtccagagaacacagtggcctatgaattaactgatcatgttaataaagacaaatggggaaatatcattgttgtttataacccaaataaaactgcagcaaccattaatttgccgagcgggaaatgggcaatcaatgctacgagcggtaaggtaggagaatccacccttggtcaagcagagggaagtgttcaagtcccaggtatatctatgatgatccttcatcaagaggtaagcccagaccacggtaaaaagtaa

SEQ ID NO: 16 - protein, 861 aa, artificial sequence
  Synthetic Construct
MSLIRSRYNHFVILFTVAIMFLTVCFPAYKALADSTSTEVIVHYHRFDSNYANWDLWMWPYQPVNGNGAAYEFSGKDDFGVKADVQVPGDDTQVGLIVRTNDWSQKNTSDDLHIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIQVGAAGYEAVNLIPRNVLNLPRYYYSGNDLGNVYSNKATAFRVWAPTASDVQLLLYNSETGPVTKQLEMQKSDNGTWKLKVPGNLKNWYYLYQVTVNGKTQTAVDPYVRAISVNATRGMIVDLEDTNPPGWKEDHQQTPANPVDEVIYEVHVRDFSIDANSGMKNKGKYLAFTEHGTKGPDNVKTGIDSLKELGINAVQLQPIEEFNSIDETQPNMYNWGYDPRNYNVPEGAYATTPEGTARITQLKQLIQSIHKDRIAINMDVVYNHTFNVGVSDFDKIVPQYYYRTDSAGNYTNGSGVGNEIATERPMVQKFVLDSVKYWVKEYHIDGFRFDLMALLGKDTMAKISKELHAINPGIVLYGEPWTGGTSGLSSDQLVTKGQQKGLGIGVFNDNLRNALDGNVFDSSAQGFATGATGLTDAIKNGVEGSINDFTSSPGETINYVTSHDNYTLWDKIALSNPNDSEADRIKMDELAQAVVMTSQGVPFMQGGEEMLRTKGGNDNSYNAGDTVNEFDWSRKAQYPDVFNYYSGLIHLRLDHPAFRMTTANEINSHLQFLNSPENTVAYELTDHVNKDKWGNIIVVYNPNKTAATINLPSGKWAINATSGKVGESTLGQAEGSVQVPGISMMILHQEVSPDHGKK

SEQ ID NO: 17 - DNA, 48 bp, artificial sequence
  FORWARD primer
ttgcttttagttcatcgatagcatcagcagattctacctcgacagaag

SEQ ID NO: 18 - DNA, 36 bp, artificial sequence
  REVERSE primer
ttattgattaacgcgtttactttttaccgtggtctg